(19) United States
(12) Patent Application Publication
(10) Pub. No.: US 2016/0331368 A1
(43) Pub. Date: Nov. 17, 2016

(54) SUTURE ANCHOR

(71) Applicant: Jonathan Ticker, Brookville, NY (US)

(72) Inventor: Jonathan Ticker, Brookville, NY (US)

(21) Appl. No.: 15/223,633

(22) Filed: Jul. 29, 2016

Related U.S. Application Data
(63) Continuation of application No. PCT/US2015/013950, filed on Jan. 30, 2015.
(60) Provisional application No. 61/934,623, filed on Jan. 31, 2014.

(51) Int. Cl. A61B 17/04

(52) U.S. Cl. CPC A61B 17/0401; A61B 2017/044; A61B 2017/0414; A61B 2017/0464

(57) ABSTRACT

The present disclosure is directed to suture anchors, and techniques for using the same, that may lead to greater patient recovery than previously obtained. In some embodiments, one or more lengths of suture are fixed relative to an anchor device. Additional lengths of suture may be sliding relative to the anchor device. The sutures may be placed in a strategic manner to minimize the stress concentration and maximize tissue compression throughout the soft tissue. The suture anchors and techniques described herein may eliminate the need to tie knots, thus adding to the security of the procedure while also reducing operative time. The suture anchors and techniques may also reduce and/or eliminate the potential failures after rotator cuff repair.

### CROSS-REFERENCE TO RELATED APPLICATIONS

This application is a continuation of International Patent Application No. PCT/US2015/013950, filed on 30 Jan. 2015, which claims the benefit of U.S. Provisional Patent Application No. 61/934,623, filed on 31 Jan. 2014, entitled “SUTURE ANCHOR.” Each application mentioned above is hereby incorporated by reference in its entirety.

### BACKGROUND

1. Field

The present disclosure is generally directed to suture anchors and surgical procedures employing the same. More particularly, the present disclosure relates to improved suture anchors and techniques that may eliminate knot tying, reduce surgical procedure times, and/or improve overall patient recovery—especially in the context of rotator cuff repairs.

2. Description of the Related Art

Procedures for repairing soft tissues may employ a combination of sutures and suture anchors. For example, suture anchors are commonly used in the shoulder and knee regions for reattaching ligaments and tendons. Surgeons commonly use one or more suture knots in such procedures where soft tissues are reattached to bones. As the soft-tissue pathology has wide variability, there is a need for devices and techniques with adaptability.

### SUMMARY

The devices, systems, and methods of the present disclosure have several features, no single one of which is solely responsible for its desirable attributes. Without limiting the scope of this invention as expressed by the claims which follow, its more prominent features will now be discussed briefly. After considering this discussion, and particularly after reading the section titled “Detailed Description of Certain Embodiments,” one will understand how the features of this disclosure provide several advantages over other suture anchors and surgical technics employing the same.

The present disclosure is directed to novel suture anchors, and techniques for using the same, that can provide improved results in procedures that include attaching soft tissue to bone.

In some embodiments, a suture anchor can comprise an anchor body. The anchor body may include at least one bone fixation member. Three or more lengths of suture may be coupled to the anchor body. At least one of the lengths of suture may be fixed relative to the anchor body. In some aspects, at least two of the three or more lengths of suture are slideable relative to the anchor body. In some aspects, at least one length of suture is fixed relative to the anchor body with a knot that contacts at least a portion of the suture anchor. The three or more lengths of suture may all be fixed relative to the anchor body. In one aspect, four length of suture are coupled to the anchor body and the four lengths are fixed relative to the anchor body.

In some embodiments a suture anchor can comprise an anchor body. Three or more lengths of suture may be coupled to the anchor body. At least one of the lengths of suture may be fixable relative to the anchor body after or during insertion of the anchor body into bone. The anchor may include a bone fixation member. In some aspects, a freely sliding suture may be positioned such that it becomes compressed between the anchor body and bone upon insertion of the anchor body into bone, thereby creating two lengths of suture fixed relative to the anchor body. In some aspects, a freely sliding suture may be fixed between two or more portions of an anchor or portion thereof.

In some embodiments, a method of attaching soft tissue to bone may include inserting a first anchor into bone. The first anchor may have three or more lengths of suture extending therefrom. The three or more lengths of suture may be coupled to the first anchor. In some aspects, at least one of the lengths of suture coupled to the first anchor is fixed relative to the first anchor. The method may further include passing the three or more lengths of suture coupled to the first anchor through the soft tissue. At least one or more of the lengths of suture may be passed through the soft tissue at a first location that is different from a second location where at least one or more other lengths of suture coupled to the first anchor are passed through the soft tissue. The method may further include securing the three or more lengths of suture coupled to the first anchor to one or more additional anchors positioned beyond an edge of the soft tissue.

In some aspects, the methods disclosed herein may also include inserting a second anchor into bone. The second anchor may have three or more lengths of suture extending therefrom that are coupled to the second anchor. At least one of the lengths of suture coupled to the second anchor may be fixed relative to the second anchor. The method may include passing the three or more lengths of suture coupled to the second anchor through the soft tissue. At least one or more of the lengths of suture coupled to the second anchor may be passed through the soft tissue at a third location different from a fourth location where at least one or more other lengths of suture coupled to the second anchor are passed through the soft tissue. The method may also include securing three or more lengths of suture coupled to the second anchor to the one or more additional anchors positioned beyond the edge of the soft tissue. In some aspects, no knots are tied above the soft tissue. In some aspects, each length of suture is passed through a separate location in the soft tissue.

In some embodiments, a method of attaching soft tissue to bone may include inserting a first anchor into bone at a position that will be underneath at least a portion of soft tissue after completion of the method. The first anchor may have three or more lengths of suture extending therefrom. The portion of soft tissue may have a top side and an underside. The method may include fixing at least one of the lengths of suture relative to the first anchor before, after, or while the anchor is inserted into the bone. The method may include passing the three or more lengths of suture up through the underside of the soft tissue. At least one or more of the lengths of suture may be passed up through the underside of the soft tissue at a first location that is different from a second location where at least one or more other lengths of suture are passed through the soft tissue. The method may also include securing the one or more lengths of suture coupled to the first anchor to one or more additional anchors positioned beyond an edge of the soft tissue. In some aspects, no knots are tied above the top side of the soft tissue. In some aspects, the fixing of at least one of the lengths of suture to the first anchor occurs after the anchor is inserted into the bone and may comprise tying a knot at a position that will be underneath at least a portion of soft tissue after completion of the method. In some embodiments, all knots are located at positions that will be underneath a portion of soft tissue after completion of the method. In other embodiments, one or more knots are located above the soft tissue after completion of the method.

In some embodiments, a method of attaching soft tissue to bone may include inserting a first anchor into a bone at a position that will be underneath at least a portion of a soft tissue after completion of the method. The first anchor may have at least a first suture length and a second suture length extending therefrom. At least one of the suture lengths may be fixed relative to the first anchor. In some aspects, the method includes fixing at least one of the suture lengths such that the at least one suture length becomes fixed relative to the first anchor. In some aspects, at least one suture may move relative to the anchor while at least one other suture is fixed relative to the anchor. The method may also include passing the first suture length through an underside of the soft tissue at a first location and passing the second suture length through the underside of the soft tissue at a second location that is different from the first location. The method may include inserting a second anchor into the bone at a position that is not underneath the soft tissue and securing at least the first suture length to the second anchor.

### BRIEF DESCRIPTION OF THE DRAWINGS

These and other features, aspects, and advantages of the invention disclosed herein are described below with reference to the drawings of certain embodiments, which are intended to illustrate and not to limit the invention. Additionally, from figure to figure, the same reference numerals have been used to designate the same components of an illustrated embodiment. The drawings disclose illustrative embodiments and particularly illustrative implementations in the context of rotator-cuff repair. They do not set forth all embodiments. Other embodiments may be used in addition to or instead. Conversely, some embodiments may be practiced without all of the details that are disclosed. The following is a brief description of each of the drawings.

FIG. 1 depicts on embodiment of an anchor device, with 4 lengths of fixed suture, each of a different color or pattern.

FIG. 2 depicts an example of a medium tear of the rotator cuff in a right shoulder as viewed from lateral.

FIG. 3 depicts the shoulder of FIG. 2 with two medial row anchors inserted, each with 3 fixed lengths of suture.

FIG. 4 depicts the shoulder of FIGS. 2 and 3 with 6 lengths of suture passed through the soft tissue, spaced from anterior to posterior.

FIG. 5 depicts a right shoulder as viewed from anterior with the supraspinatus and a portion of the upper subscapularis retracted medially, which allows for visualization of example medial and lateral bone hole positions.

FIG. 6 depicts the shoulder of FIGS. 2, 3, and 4 with completed repair of the rotator cuff tendon to the greater tuberosity of the proximal humerus.

FIG. 7 is similar to FIG. 3 and depicts an example of a smaller tear of the rotator cuff in a right shoulder as viewed from lateral with one medial anchor having four lengths of suture coupled thereto. In some aspects, two of the suture lengths may be fixed relative to the anchor while the other two suture lengths may slide with respect to the anchor.

FIG. 8 depicts the shoulder of FIG. 7 with four lengths of suture passed through the soft tissue, spaced from anterior to posterior.

FIG. 9 depicts the shoulder of FIG. 8 with a completed repair of the rotator cuff tendon to the greater tuberosity of the proximal humerus using a single lateral anchor.

FIG. 10 depicts an embodiment of an anchor device that is similar to the embodiment shown in FIG. 1, with 4 lengths of fixed suture, except that the 4 lengths of suture have one of two color patterns.

FIGS. 11-13 are similar to FIGS. 3, 4, and 6-9 and depict a method of using the suture anchor of FIG. 7.

FIG. 14-15 depicts another embodiment where two lengths of suture are provided by passing a suture through a suture anchor and tying a knot.

FIGS. 16-19 are similar to FIGS. 3, 4, 6-9, and 11-13 and depict a method of using suture anchors provided in FIG. 10.

FIG. 20 is a photograph of a comparative construct that was prepared according to the transosseous-equivalent-type suture-bridge technique with two medial-row anchors and two lateral row knotless anchors.

FIG. 21 is a photograph of a comparative construct that was prepared using a speed-bridge-type suture tape technique with two medial row anchors and two lateral row knotless anchors.

FIG. 22 is a photograph of a comparative construct that was prepared according to the technique disclosed herein with two medial row anchors and two lateral row knotless anchors.

### DETAILED DESCRIPTION OF THE PREFERRED EMBODIMENTS

While a variety of techniques for re-attaching soft tissue to bone exist, new techniques are needed to simplify procedures, decrease surgery time, increase surgery success rates, and enhance patient safety. In the context of rotator cuff repair, the surgical technique should restore the anatomic footprint, resist gap formation and cyclic elongation, have the highest ultimate tensile strength necessary (or ultimate load to failure necessary), and should resist more cycles prior to failure.

Arthroscopic knot tying, even in the hands of experienced arthroscopic surgeons, can be variable and/or can adversely affect the security of the repair construct. Accordingly, the suture anchors and techniques described herein can eliminate the need to tie knots, thus adding to the security of the procedure while also reducing operative time. The suture anchors and techniques may also reduce and/or eliminate the potential failures after rotator cuff repair. Moreover, the anchors and techniques may lead to greater patient recovery than previously obtained. In some embodiments, the present invention uses one or more lengths of suture that are fixed to an anchor device and places these sutures in a strategic manner to minimize the stress concentration and maximize tissue compression throughout the soft tissue. Additional lengths of suture may be sliding relative to the anchor device.

Some embodiments include an anchor that can be fixed into bone, having multiple lengths of sutures independent of each other, with each length of suture coupled to and extending from the anchor body. Anchors or suture anchors are devices which can be used to attach soft tissue to bone. This may be achieved by tying one end of a suture to soft tissue and the other end to a device which anchors the suture to the bone. Alternatively, two or more anchors may be used with suture passing over the soft tissue in between the anchors. Anchors come in any number of configurations and may be, for example, deployable, bioresorbable, press-fit, screw-in, or screw-in with a washer. Anchors may be made from one or more of titanium, coated titanium, nitinol, stainless steel, plastics, suture materials, and the like. In some cases, anchors are made from biodegradable materials.

As used herein, “anchor body” refers to the anchor excluding the lengths of suture, and can include multiple parts, including a suture eyelet, a post, or other features. In some embodiment, the “anchor body” is a multiple-part anchor, such as a two-part anchor, that contains multiple independent components that come together during use to form the entire anchor body. The anchor body may include a screw or screw-like portion. In some configurations, the anchor body includes a press fit or interference fit portion.

As used herein, when a length of suture is “coupled” to the anchor body, it is meant that the suture is connected to the anchor body in some fashion, but not necessarily fixed relative to the anchor body. Thus, a length of suture coupled to the anchor body may be moveable (e.g., slideable) relative to the anchor body or may be fixed relative to the anchor body.

In some embodiments, at least one length of suture is non-sliding relative to the anchor. In some embodiments, two or more lengths of suture are suture limbs that are part of a suture that is slideable relative to the anchor body. In various non-limiting embodiments, the point of attachment of the lengths of suture, which in the case of a slideable suture is the point of sliding, is beside, under, above, or within the anchor body.

The anchor body can be of any configuration that allows it to be placed securely into bone. Thus, for example, the anchor can be secured in the bone using one or more bone fixation members. Non-limiting examples of bone fixation members include threads, structures facilitating an interference fit (e.g., ridges or texture surfaces), expansion members, spikes or other sharpened members, etc. Securing the anchor bone in bone may involve one action by the surgeon, such as screwing the anchor into the bone, tapping the anchor into the bone, deploying an expansion member after insertion of the anchor into bone, or rotation of the anchor body itself after insertion into bone. The number of lengths of suture can vary, and, for example can be 3, 4, 5, 6, 7, 8, or more lengths of suture coupled to the anchor. The fixed lengths of suture can be attached via knots, crimping, adhesive, or other suitable means. The slideable lengths of suture can pass through an eyelet or around a post in the anchor, for example. In some embodiments, each length of suture can be part of a longer continuous suture. For example, in one embodiment, a suture is threaded through an aperture on the anchor and knotted to form two suture limbs fixed relative to the anchor. In some embodiments, knots used to fix suture limbs may contact the anchor body or another member connected to the anchor body. In some embodiments, the knots may form a suture loop that passes through the anchor. The sutures may comprise any suitable suture material and may be any suitable length and/or thickness. In some embodiments, one or more sutures are impregnated and/or coated with one or more growth factors, biological factors, and/or the like. In some embodiments, one or more sutures are impregnated and/or coated with one or more antibiotics. In some embodiments, one or more sutures are made from absorbable suture materials. Any combination of suture material may be used with the same anchor. For example, the set of sutures that are used may vary in thickness, length, and/or material.

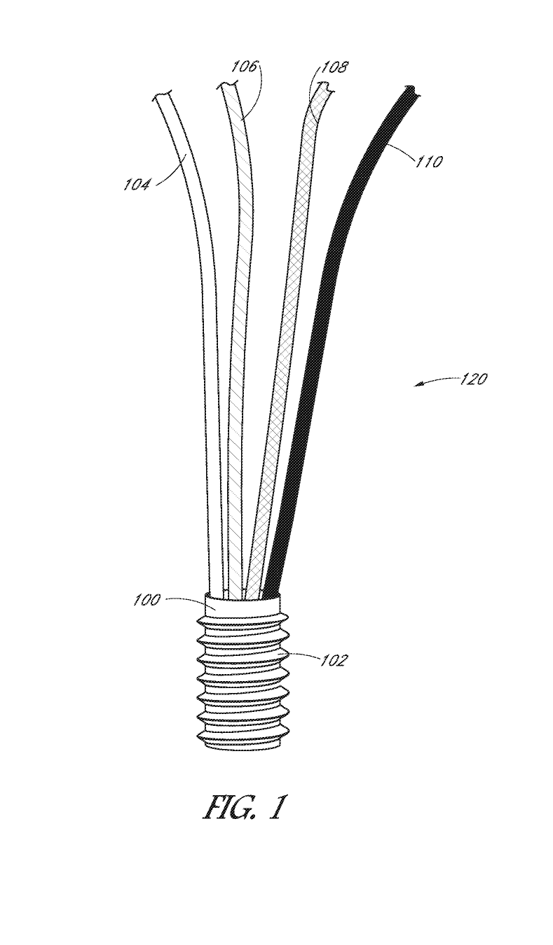

One embodiment of the anchor described above is depicted in FIG. 1. The embodiment of the anchor 120 shown in FIG. 1 includes an anchor body 100 that comprises threads 102 to secure the anchor body 100 into bone. While the depicted embodiment includes a threaded anchor body, any suitable bone fixation structure may be employed. Four fixed lengths of suture 104, 106, 108, and 110 extend from the interior of the anchor body. In some embodiments, each length of suture has a different color or pattern to aid the surgeon in visually distinguishing the sutures. However, in some embodiments, each length of suture has the same color or pattern.

While FIG. 1 illustrates four lengths of suture that each have one end fixed to the anchor, other configurations are contemplated. For example, in some embodiments, one or more of sutures may be slideable with respect to the anchor. In some embodiments, the sutures and/or suture anchor may be configured to be moved relative to the anchor and then fixed either by a knot or other technique. For example, the anchor may include one or more movable securements that may move from an open position to a closed position. In the open position, one or more suture may be moved and/or slid with respect to the anchor. In the closed position however, one or more sutures may be fixed relative to the anchor. The moveable securement may include, for example, a clamp, clasp, compression member, screw, locking member, movable pin, and the like. In other embodiments, the slideable suture may become fixed while the anchor is inserted into bone, such as be compressing the suture between the side of the anchor and the bone.

Some embodiments include use of the suture anchor described above to secure soft tissue to bone. In some embodiments, this is achieved in a knotless fashion. In various embodiments, the procedure can be performed arthroscopically or using an open approach. In one example of how the device is used with this method, the technique includes reattaching tendon to bone, such as to repair a rotator cuff tendon tear in the shoulder. Non-limiting other uses include soft tissue repair elsewhere in the body, such as foot & ankle, knee, elbow, and hip, for example. The following description and examples illustrate preferred embodiments of the present suture anchor devices disclosed in the context of use in rotator cuff repair.

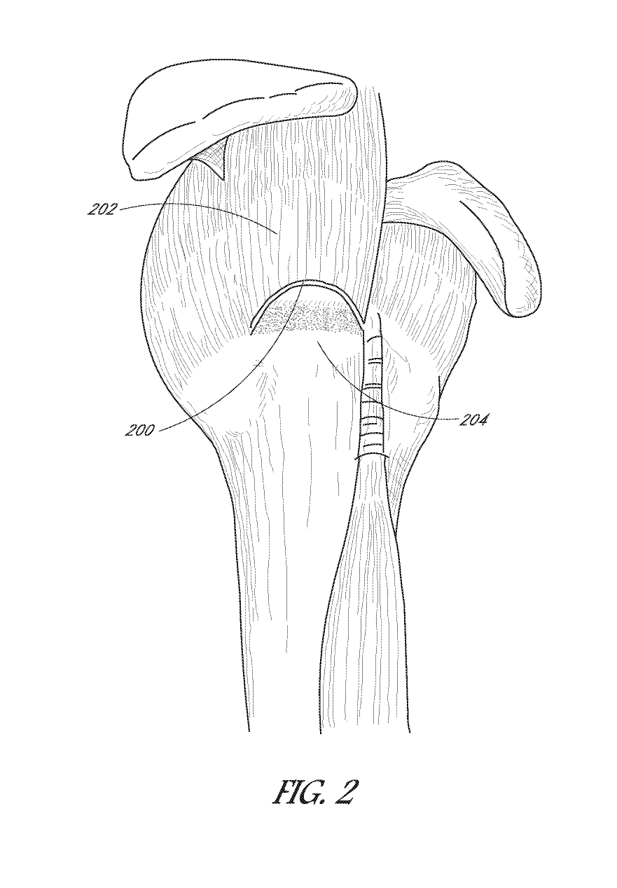

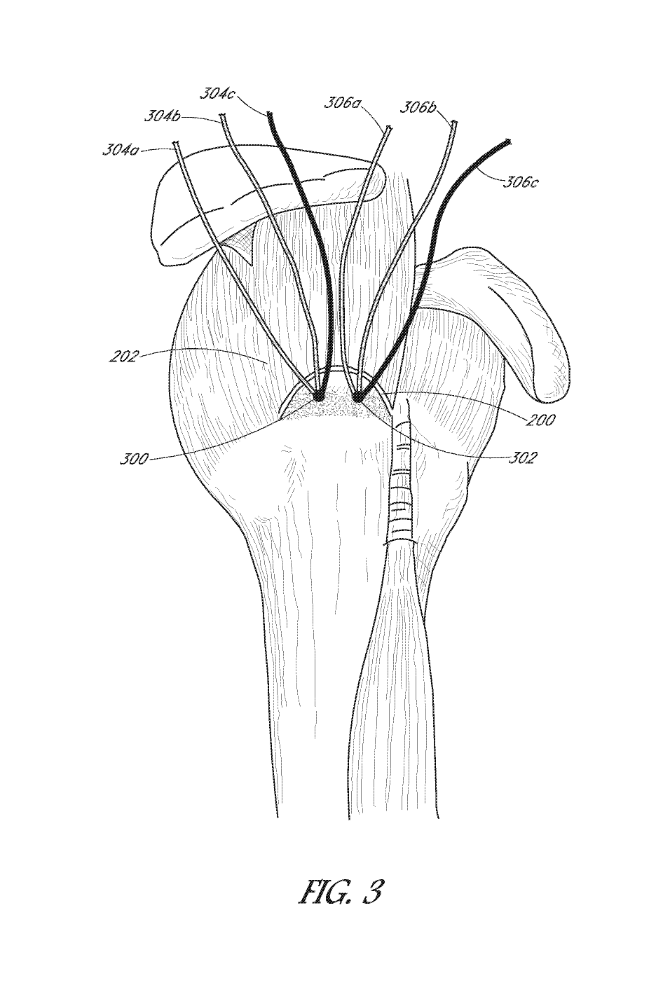

As one non-limiting example, repair of a medium-sized crescent-shaped rotator cuff tear can be achieved using two of the anchors described above. FIG. 2 depicts the lateral view of a right shoulder having a medium tear of the rotator cuff tendon 202 having a tendon tear edge 200. The bone of the greater tuberosity 204 is exposed and prepared. As depicted in FIG. 3, two anchors 300 and 302 as described above are inserted medially on the footprint from which the tendon tore. These anchors are referred to as the medial row of anchors. In some embodiments, if the tear size is approximately 2 cm to 2.5 cm in size, each anchor device could have 3 fixed lengths of suture, for a total of 6 lengths of suture 304a, 304b, 304c, 306a, 306b, and 306c available for repair. As depicted, each length of suture can have a different color or pattern to aid the surgeon in distinguishing the sutures.

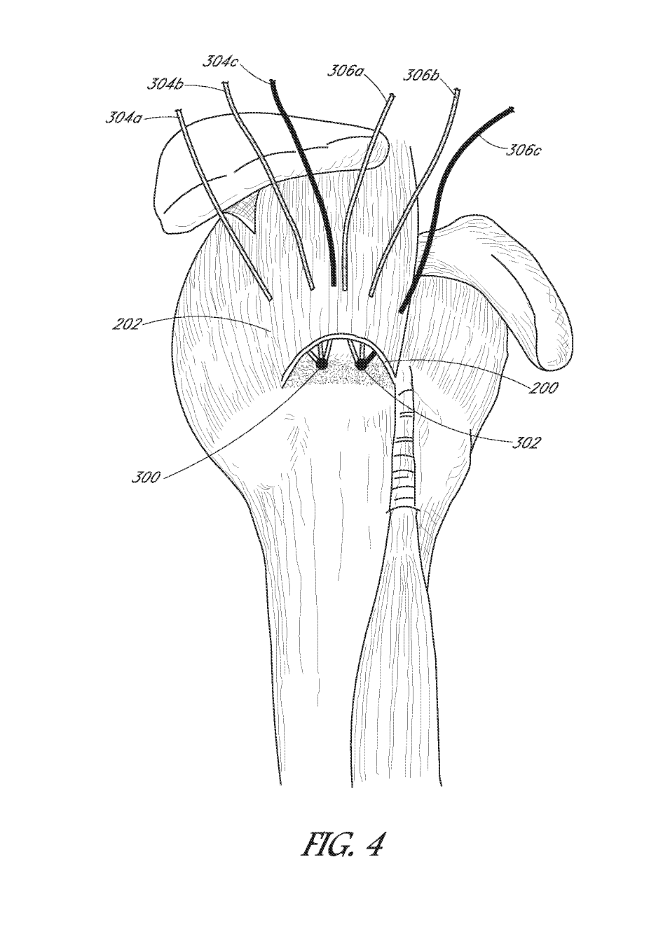

As depicted in FIG. 4, each of the 6 lengths of suture 304a, 304b, 304c, 306a, 306b, and 306c are passed through the rotator cuff tendon 202 an optimal distance medial to the tendon tear edge 200. In some embodiments, as depicted, the locations of the length of suture passing through the rotator cuff tendon 202 are spaced apart from each other anterior to posterior. In some embodiments, the spacings are approximately equal. In various embodiments, the spacings range from 2 mm to 3 cm, from 2 mm to 2.5 cm, from 2 mm to 1.5 cm, from 2 mm to 1 cm, from 3 mm to 1 cm, from 3 mm to 8 mm, from 5 mm to 2.5 cm, from 8 mm to 2.5 cm or from 1 cm to 2.5 cm. The method for passing the 6 lengths of suture through the tendon can vary, but in this example the sutures 304a, 304b, 304c, 306a, 306b, and 306c are passed up through the tendon 202 separately. In some alternative embodiments, two or more of the multiple lengths of suture available from the medial row of anchors may be passed through the tendon 202 together, such that although at least some lengths of suture are spaced apart from at least some other lengths of suture, the two or more lengths of suture pass through the tendon 202 in the same location.

Next, alternating lengths of suture can be paired and retrieved together through a portal. For example, in one embodiment lengths of suture 304b, 306a, and 306c are retrieved through a portal and “parked” outside the portal. The remaining lengths of suture 304a, 304c, and 306b are similarly retrieved together and “parked” outside a portal, which may be the same or different from the portal used to retrieve lengths of suture 304b, 306a, and 306c.

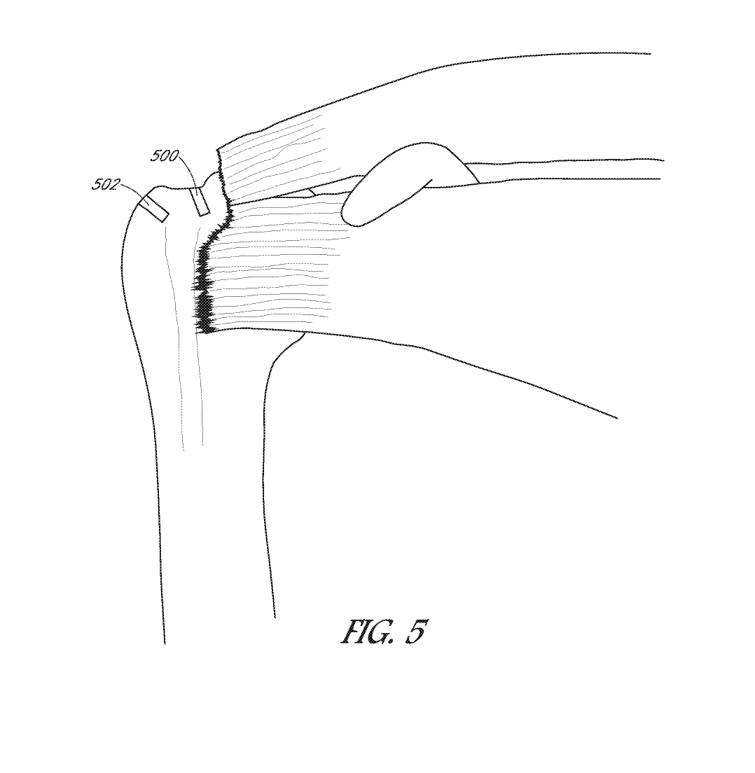

In the depicted embodiments, the repair is completed using one or more anchors placed beyond the edge of the tendon tear. This second row of anchors is referred to as the lateral row in the setting of a rotator cuff tear repair. FIG. 5 depicts the right shoulder as viewed from anterior, showing the supraspinatus and a portion of the upper subscapularis retracted medially, which allows for visualization of possible bone hole positions medially 500 and laterally 502 for the medial and lateral anchors, respectively. Any suitable bone anchor may be used in the lateral row as long as it is configured to have suture secured thereto. Suture may be secured to the lateral row anchor by any suitable means, such as by tying knots or any one of a number of knotless suture capture techniques. Any number of lateral row anchors may be used, as determined by the surgeon. In the depicted embodiment, two lateral anchors are utilized. The lateral row anchors may be placed at any time prior to their use to secure the lengths of suture. In some embodiments, the lengths of suture are coupled to the lateral anchor prior to their insertion and suture fixation.

As depicted in FIG. 6, the first set of paired-off lengths of suture 304b, 306a, and 306c are brought over the cuff tendon (202 in FIG. 4) and tendon tear edge (200 in FIG. 4) and then secured to the anterior lateral anchor 602. Next, the second set of paired-off lengths of suture 304a, 304c, and 306b are brought over the cuff tendon (202 in FIG. 4) and tendon tear edge (200 in FIG. 4) and the second set of lengths of suture and secured to the posterior lateral anchor 600.

In one example, one paired-off set of lengths of suture are placed into a portion of a lateral anchor fixation device used for knotless repair. This portion of the anchor with the sutures within it is inserted into the bone of the greater tuberosity inferior to the lateral edge of the greater tuberosity. In certain instances, a bone hole is created before the portion of the anchor is inserted. The lengths of suture can then be tensioned individually to compress the tendon (to just the right amount as determined by the surgeon) onto the prepared bony bed of the sulcus of the greater tuberosity, and then the sutures are fixed into the bone by inserting the second portion of the anchor into the bone flush with or recessed to the bone cortex. These steps of the repair can be all done arthroscopically and knotlessly, but could also be done in an open fashion. The above steps can then be repeated with the other set of paired-off lengths of suture. The result is a completed rotator cuff repair using all 6 lengths of suture.

In the circumstance of a smaller tear, the procedure could be completed with one medial row anchor and one or two lateral row anchors, also in a truly knotless connected double row rotator cuff repair. In some embodiments, two medial row anchors and one or two lateral row anchors may be employed. In another embodiment, one medial row anchor having both fixed and sliding sutures, and one lateral row anchor may be employed. The options could be more varied, depending upon the tear pattern the surgeon encounters. A large rotator cuff tear could be repaired with three medial row anchors, each with 3 lengths of suture, and two or three or more lateral row anchors. Or, this large rotator cuff tear could be repaired with two medial row anchors, each with 4 lengths of suture, and three lateral row anchors, as other examples of the varied options. It is to be understood that the sutures need not to be deployed to the lateral anchors in an even or symmetrical fashion. As a non-limiting example, three medial row anchors each having three lengths of suture may be used with two lateral row anchors. Thus, one lateral row anchor may be used to capture four lengths of suture while the other lateral row anchor may be used to capture the remaining five lengths of suture.

In general, any combination of numbers of medial row anchors, lateral row anchors, and lengths of suture on each medial row anchor can be completed. For example, the number of medial row anchors can be 1, 2, 3, 4, or more anchors. The number of lateral row anchors can be 1, 2, 3, 4, or more anchors. As described above, the number of lengths of suture coupled to the medial row anchors can be 3, 4, 5, 6, 7, 8, or more lengths of suture. Any combination of the above numbers can be used together.

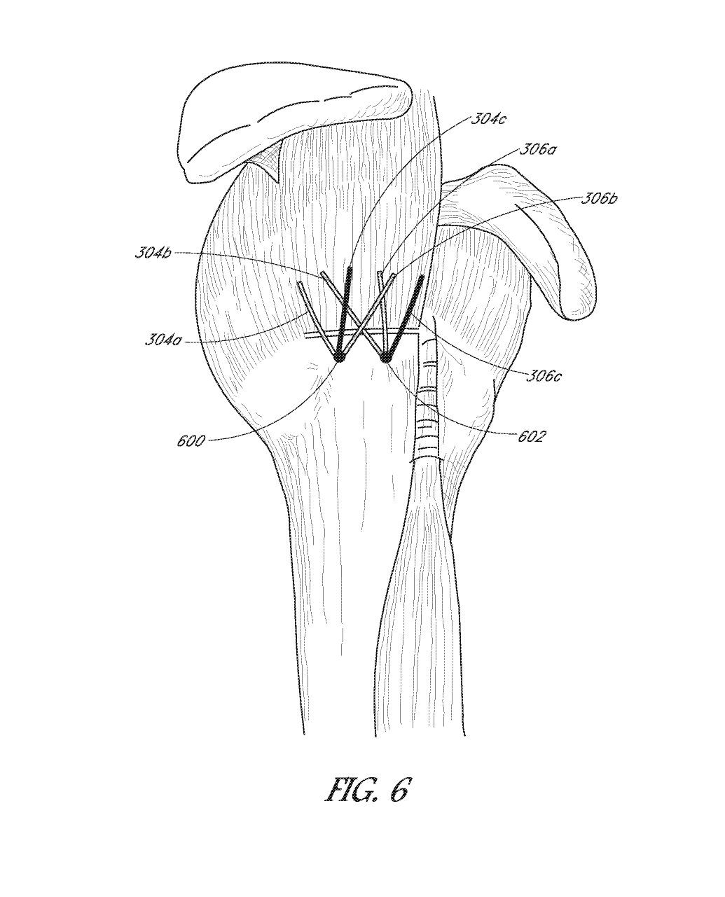

FIGS. 7-9 depict a procedure using one medial row anchor and one lateral row anchor, in the context of a knotless rotator cuff repair surgery. Such an implementation may be useful in repairing, for example, smaller sized tears. As shown in FIG. 7, a single anchor 125 may be secured to the bone in an area that will be located underneath at least a portion of the separated tendon 202 after the repair is complete. FIG. 7 illustrates the single anchor 125 having four suture lengths coupled to the anchor 125. The lengths of suture are labeled from left to right: 114, 118, 118, 114. As shown in FIGS. 7-9, suture lengths 114 have a different color or color pattern than suture lengths 118. For example, suture lengths 114 may be two limbs from a first suture having a first color or pattern that is coupled to the anchor 125 and suture lengths 118 may be two limbs from a second suture having a second color or pattern that is coupled to the anchor 125. In the illustrated example, suture lengths 114 are darker in color than suture lengths 118 and are arranged as the two outside most suture lengths. The two lighter colored suture lengths 118 are arranged as the two inner suture lengths. In an alternative embodiment, 3 or 4 different colors or patterns may be used for the suture lengths 114, 118. One or more of the suture lengths 114, 118 may be fixed relative to the anchor 125. In some embodiments, one or more of the suture lengths 114, 118 are slideable relative to the anchor 125. The suture lengths 114, 118 may be fixed to the anchor 125 before, during, and/or after the anchor 125 is secured to the bone. More or less than four suture lengths may be coupled to the anchor 125.

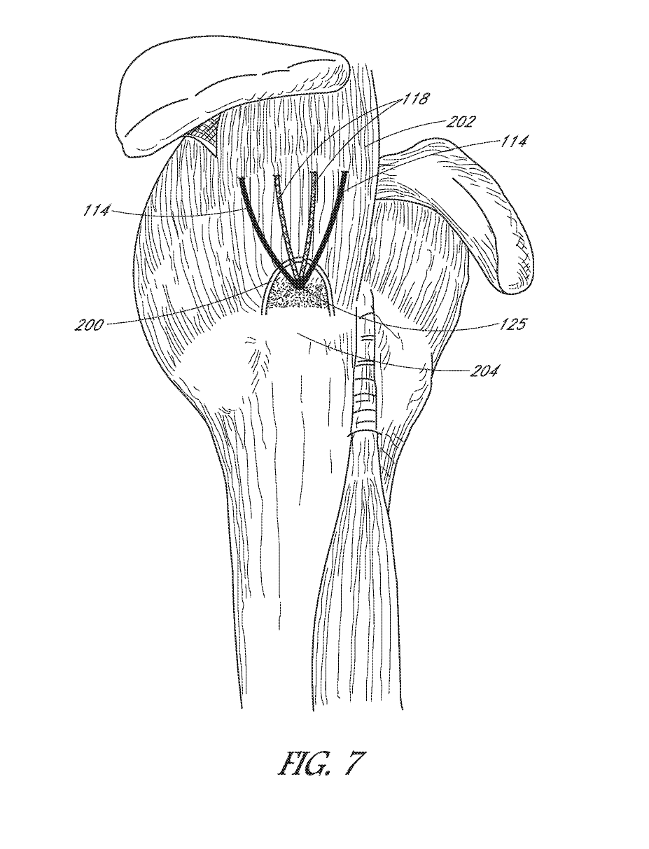

Turning to FIG. 8, the suture lengths 114, 118 extending from the anchor 125 are passed up through the underside of tendon 202. As shown in FIG. 8, each length of suture passes up through the underside of tendon 202 at a different location. In other words, the suture lengths 114, 118 are passed through the underside of the tendon 202 at different locations that are spaced apart from one another by a distance. In FIG. 8, the locations where the suture lengths 114, 118 are passed through are generally evenly spaced apart along a curved arc that is roughly parallel to the tendon tear edge 200. However, the suture lengths 114, 118 may be passed through any portion or portions of the tendon 202 that the surgeon desires. For example, the locations where the suture lengths 114, 118 are passed do not need to be parallel to the tendon tear edge 200 along a single line. In various embodiments, the distances between the locations through which the sutures pass range from 2 mm to 3 cm, from 2 mm to 2.5 cm, from 2 mm to 1.5 cm, from 2 mm to 1 cm, from 3 mm to 1 cm, from 3 mm to 8 mm, from 5 mm to 2.5 cm, from 8 mm to 2.5 cm or from 1 cm to 2.5 cm.

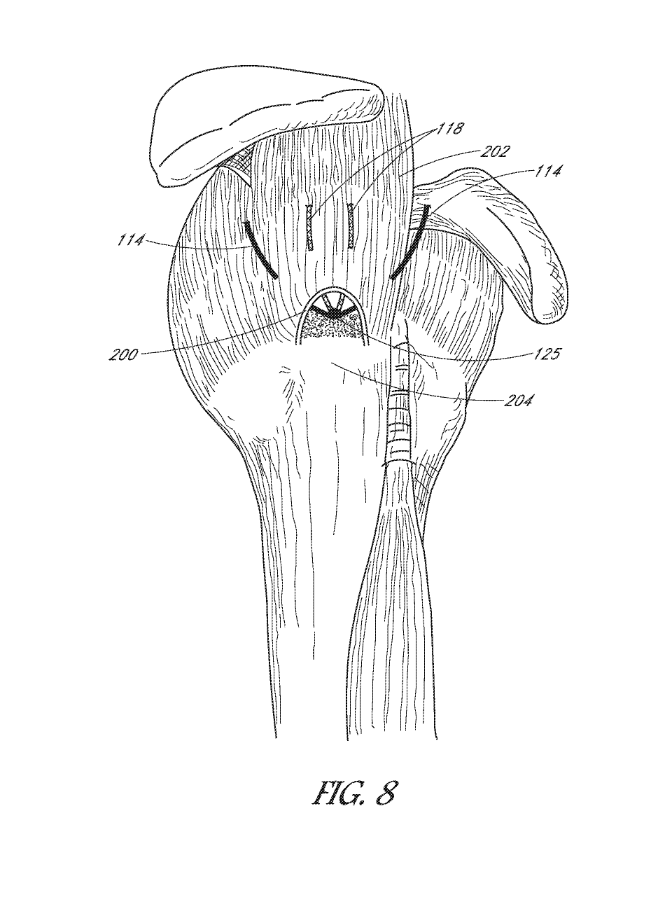

FIG. 9 depicts the completed repair. As shown in FIG. 9, the four suture lengths 114, 118, 118, and 114 are gathered and secured to a lateral anchor 300 that is secured to the bone 204 at an area beyond the tendon tear edge 200. The suture lengths 114, 118 may be secured to the lateral anchor 300 in a knotless fashion.

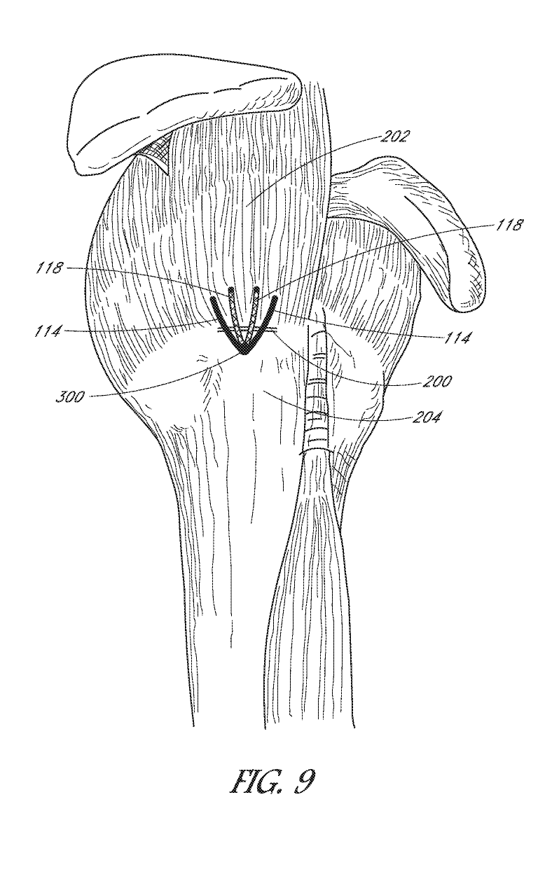

Another embodiment of the anchor described herein is depicted in FIG. 10. As shown, the anchor 130 includes an anchor body 100 having threads 102 to secure the anchor body 100 into bone. Four lengths of suture extend from the anchor body 100. As illustrated in FIG. 10, two suture lengths 108 have a first pattern and the remaining two suture lengths 106 have a second color pattern. Thus, the suture lengths alternate between a first pattern and a second pattern. In this way, a surgeon may easily distinguish suture length 106 from suture length 108. In some embodiments, suture lengths 106 are all secured to a first lateral anchor while suture lengths 108 are secured to a second lateral anchor. The suture lengths may have any number of patterns and/or colors to aid in the visualization of one or more of the suture lengths.

FIGS. 11-13 depict an example method of using two anchors 130 in a procedure to attach soft tissue to bone. As shown in FIG. 11, the tendon 202 has separated from the bone 204. Two anchors 130a, 130b have been inserted under the tendon 202 near the medial border of the tendon 202 footprint. That is to say, the two anchors 130a, 130b are secured into a portion of the bone that will be located underneath at least a portion of the separated tendon 202 after the repair is complete. The two anchors 130a, 130b are spaced apart by a distance. In some embodiments, the distance between the two anchors 130a, 130b is about 1 cm. More or less than two anchors may be used. In general, as shown in FIG. 11, at least two anchors 130a, 130b are arranged in roughly a straight line extending under the separated tendon 202. However, the anchors can be arranged in any manner desired by the surgeon. These anchors 130 may be generally referred to as the medial row of anchors.

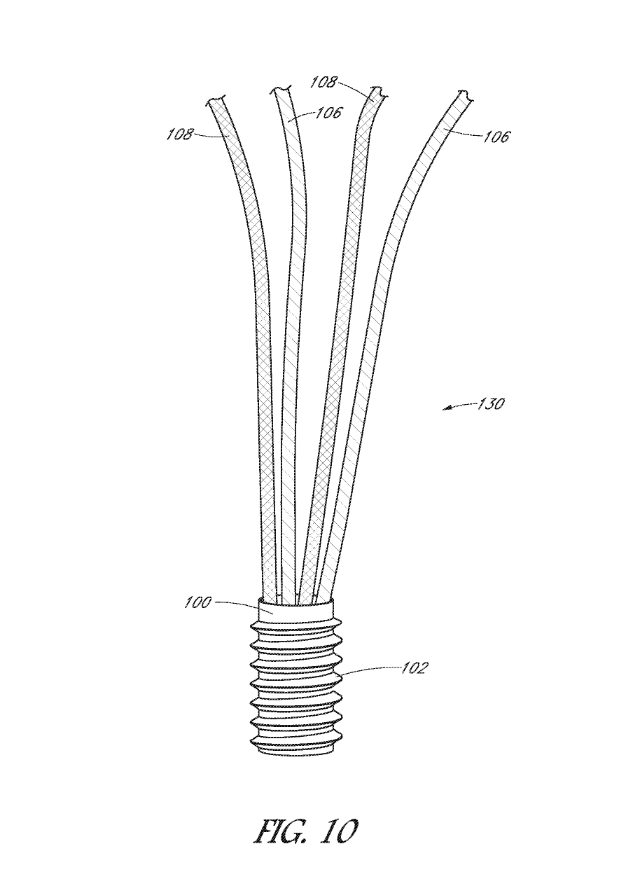

Moving on to FIG. 12, the suture lengths 106, 108 extending from the anchors 130a, 130b are passed up through the underside of tendon 202. As depicted in FIG. 12, the locations of the length of suture 106, 108 passing through the rotator cuff tendon 202 are spaced apart from each other anterior to posterior. In some embodiments, the spacings are approximately equal. But, the spacing between the locations where the lengths of suture 106, 108 are passed through may be varied and more than one suture length may pass through approximately the same location as another suture length. In various embodiments, the spacings range from 2 mm to 3 cm, from 2 mm to 2.5 cm, from 2 mm to 1.5 cm, from 2 mm to 1 cm, from 3 mm to 1 cm, from 3 mm to 8 mm, from 5 mm to 2.5 cm, from 8 mm to 2.5 cm or from 1 cm to 2.5 cm.

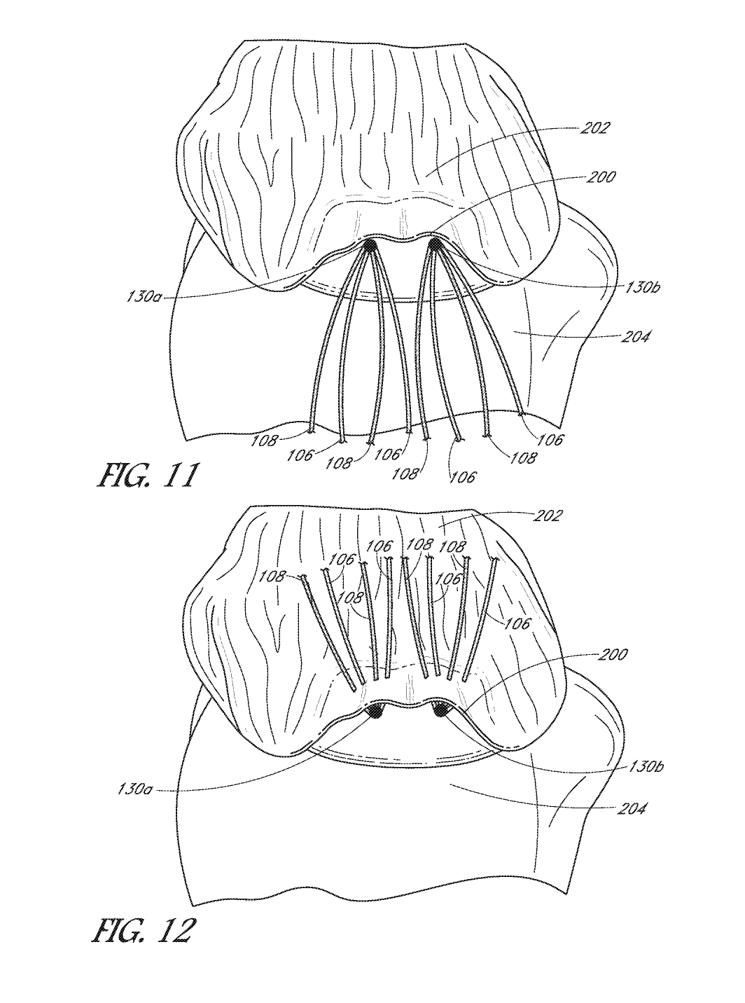

In FIG. 13, the repair is completed using one or more anchors placed beyond the edge of the tendon tear. This second row of anchors is referred to as the lateral row in the setting of a rotator cuff tear repair. As shown in FIG. 13, the lateral row of anchors includes two anchors 205a, 205b. However, more or less than two lateral anchors may be used. Suture lengths 108 can be gathered and secured in a knotless fashion to anchor 205a while suture lengths 106 can be gathered and secured in a knotless fashion to anchor 205b. As shown in FIG. 13, suture lengths 108 may overlap one or more of suture lengths 106. However, in other embodiments, suture lengths 106 overlap one or more of suture lengths 108. The result is a completed repair using eight lengths of suture.

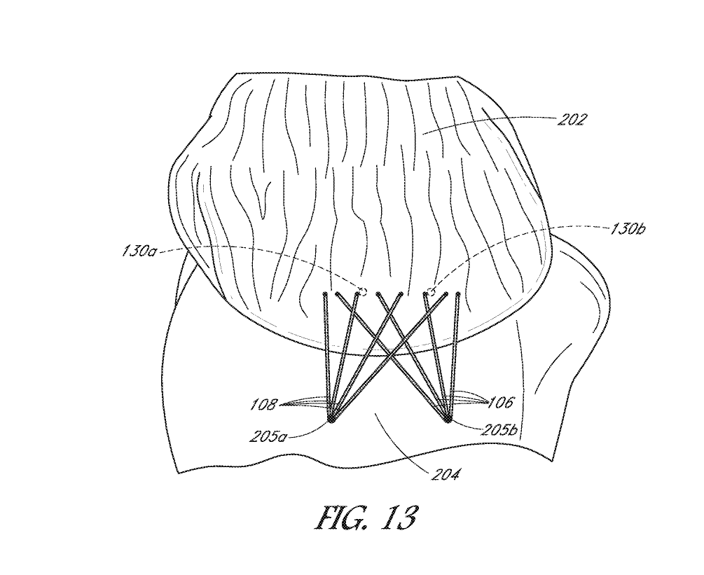

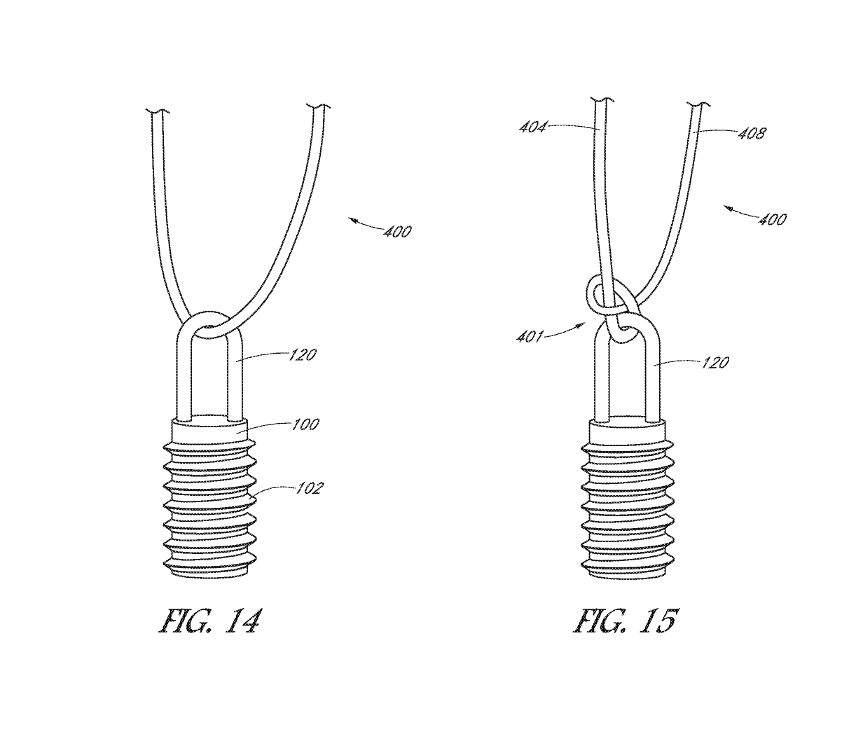

FIGS. 14-15 depict another embodiment of an anchor 400 according to the present disclosure. As shown, the anchor 400 includes an anchor body 100 having threads 102 to secure the anchor body 100 into bone. As illustrated in FIG. 14, the anchor 400 also includes an eyelet 120 having a slideable length of suture passing through the eyelet 120. Turning to FIG. 15, the slideable length of suture passing through the eyelet 120 may be tied in a knot 401 such that two suture lengths 404, 408 are fixed to the anchor 400. More or less lengths of suture may be secured to the anchor in the same or similar manner. For example, two slideable sutures may be knotted about the eyelet to form four suture lengths extending from the anchor. Furthermore, one or more suture lengths may be trimmed to reduce the number of suture lengths extending from the anchor. The knots may contact the eyelet 120, or alternatively, may be tied such that a suture loop is formed that passes through the eyelet 120. In some embodiments, one or more sutures remain slideable through the eyelet 120.

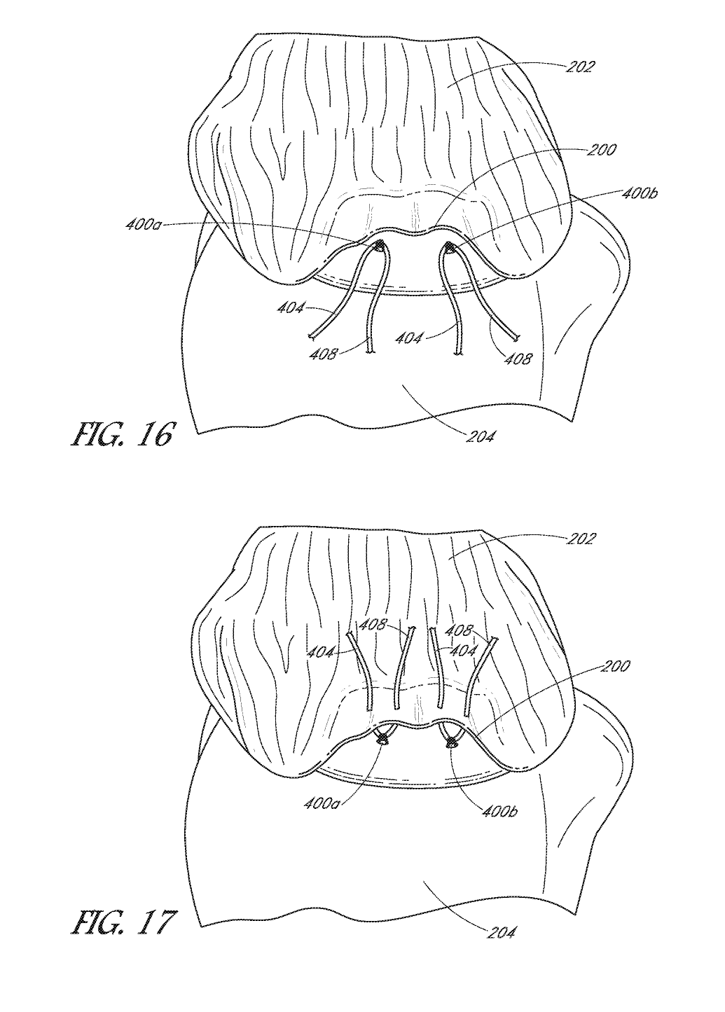

Similar to the techniques described above, FIGS. 16-19 depict an example method of using two anchors 400 in a procedure to attach soft tissue to bone. In FIG. 16, two anchors 400a, 400b are secured in a position that will be located under the soft tissue tear edge 200. The lengths of suture may be coupled to the anchor before or after the anchor is inserted into the bone. For example, in one embodiment, the anchor is first inserted into the bone in a position that will be located under the soft tissue. One or more lengths of suture may then be passed through the eyelet. One or more of these lengths of suture may then be fixed relative to the anchor. For example, at least one length of suture may be knotted in the same or similar manner as depicted in FIG. 15, above. In this way, any knotting may be performed in an area that will be under the soft tissue. Thus, in this embodiment, knotting above the soft tissue is eliminated. As discussed above, other techniques and/or structures may be used to fix one or more of the sutures to the anchor. In some embodiments, one or more sliding sutures may be changed to fixed sutures as the anchor is being inserted. In some embodiments, the anchor may be configured such that sutures can slide with respect to the anchor when the anchor is in a first position and the sutures become fixed in a second position. In some embodiments, the anchor may be configured such that the sutures are fixed to the anchor after the anchor is screwed or press-fit into the bone. In one such embodiment, the suture is secured at least in part by compression between the anchor and the side of the bone hole. In one non-limiting example, the anchor includes an eyelet that is configured to close as the anchor is inserted into bone.

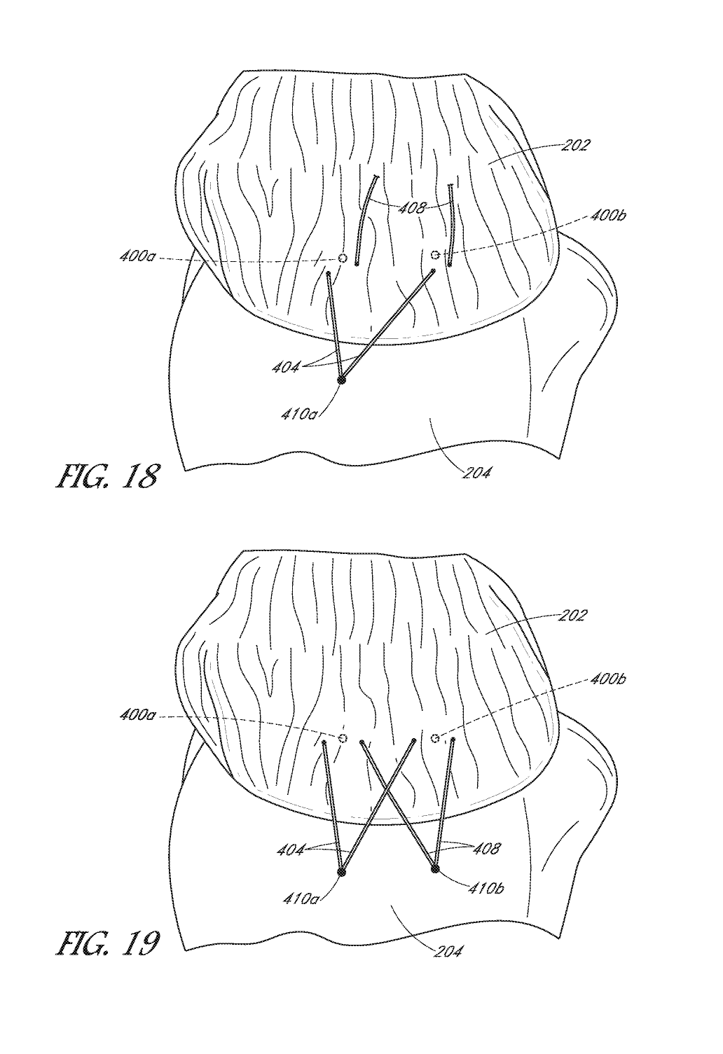

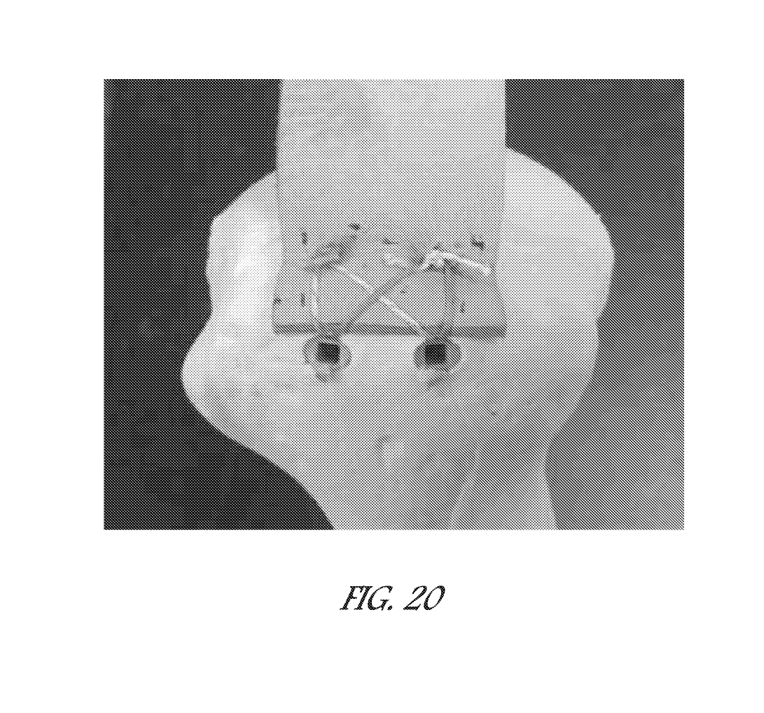

In FIG. 17, the two lengths of suture 404, 408 secured to each anchor 400a, 400b are passed up through the underside of the soft tissue 202. The lengths of suture may be passed through the soft tissue at spaced apart locations. In various embodiments, the spacings range from 2 mm to 3 cm, from 2 mm to 2.5 cm, from 2 mm to 1.5 cm, from 2 mm to 1 cm, from 3 mm to 1 cm, from 3 mm to 8 mm, from 5 mm to 2.5 cm, from 8 mm to 2.5 cm or from 1 cm to 2.5 cm. In FIG. 18, suture lengths 404 are gathered and then secured to bone 204 at location beyond the edge of the soft tissue 202 in a knotless fashion with a first lateral anchor 410a. In FIG. 19, suture lengths 408 are gathered and then secured to bone 204 at location beyond the edge of the soft tissue 202 in a knotless fashion with a second lateral anchor 410b. As shown in FIG. 19, at least one suture length 408 coupled to a first anchor 400a may overlap at least one suture length 404 coupled to a second anchor 400b. In this way, the soft tissue tear is repaired.

The above description is provided to enable any person skilled in the art to make or use embodiments within the scope of the disclosed inventions. Various modifications to these aspects will be readily apparent to those skilled in the art, and the generic principles defined herein may be applied to other aspects without departing from the scope of the disclosure. Thus, the present disclosure is not intended to be limited to the aspects shown herein but is to be accorded the widest scope consistent with the principles and novel features disclosed herein.

The following non-limiting examples are provided.

### Example 1

The following study was conducted to evaluate the comparative effectiveness of the anchors and techniques described herein in comparison with two other repair constructs in the context of a simulated rotator cuff repair procedure. The tests utilized foam models for the proximal humeral head and uniform leather straps for the tendon.

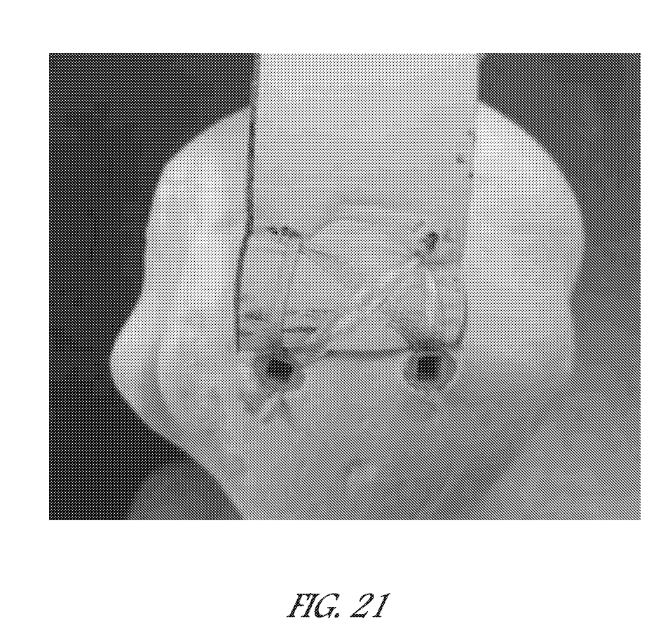

Comparative Construct 1 was prepared according to a transosseous-equivalent (“TOE”) suture-bridge-type technique with two medial row anchors and two lateral row knotless anchors. Four lengths of suture extended from each medial anchor (two sliding sutures). The sutures were passed through the tendon model and one suture was knotted above the tendon above each medial anchor. These two of the four lengths were trimmed. Comparative Construct 1 is shown in FIG. 20.

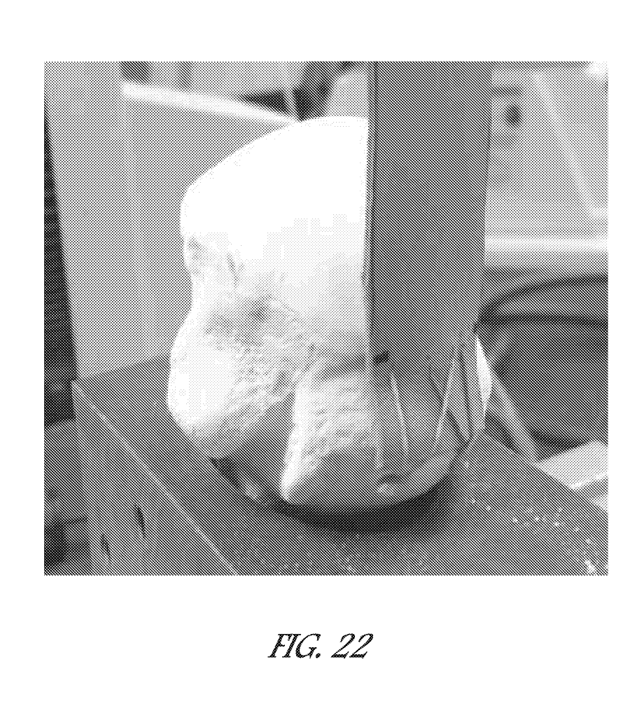

Comparative Construct 2 was prepared using a speed-bridge-type (“SB”) suture tape (using Ultratape) technique with two medial-row anchors and two lateral row knotless anchors. Fixed lengths of wider suture tape without knots were employed. Two lengths of suture extended from each medial anchor through the same location in the tendon model. Comparative Construct 2 is shown in FIG. 21.

Construct 3 was prepared according to the technique disclosed herein with two medial row anchors and two lateral row knotless anchors. Two lengths of fixed suture (#2 Ultrabraid) extended from the two medial row anchors. Construct 3 is shown in FIG. 22.

Each construct was tested three times. Gap formation under cyclic loading at 500 cycles (1 cycle/second following a preload) and ultimate construct load at failure (19.3 inch/minute following a preload), and failure mode were assessed.

The results of gap formation revealed an average gap formation of 2.6 mm (0.6 mm SD) for Construct 3, while Comparative Construct 1 was 3.7 mm (0.6 mm SD) and

Comparative Construct 2 was 4.2 mm (1.4 mm SD). The sample size was not large enough to demonstrate a statistical difference.

For ultimate failure load, the average (in pound/force) for Construct 3 was 71.6 lbf (6.8 lbf SD), while Comparative Construct 1 was 63.1 lbf (6.5 lbf SD) and Comparative Construct 2 was 62.6 lbf (4.5 lbf SD). The sample size was not large enough to demonstrate a statistical difference.

For the failure mode, the leather tore in all cases, with less catastrophic failure noted in the Construct 3.

### Example 2

A series of patients were treated according to the methods described herein. The inclusion criteria were patients who had a medium posterosuperior cuff tear repairs.

Patients with medium tear repairs who were involved in legal matters were excluded. Repairs of all other size posterosuperior tears, including partial tears, were excluded, as well as repairs of full thickness subscapularis tendon tears and revision repairs. Any repair using a technique with knots tied over the tendon and/or single row repairs were not included.

Seven patients, with a mean age of 54.3 years, who had a medium posterosuperior cuff tear repair, using the method described herein were included in the analysis. After at least a six month period, patients were assessed according to the validated SANE score. The patient is asked how the repaired shoulder feels using a scale of 0-100, with 100 being perfectly normal. The SANE score is listed below in TABLE 1 next to the patient number.

TABLE 1

| Patient Number | SANE Score |
|---|---|
| 1 | 95 |
| 2 | 95 |
| 3 | 80 |
| 4 | 80 |
| 5 | 50 |
| 6 | 90 |
| 7 | 96 |

The mean result was 83.7. After removing the outlier (Patient 5), the mean was 89.3. Patient 5 was a smoker, stopped physical therapy early, had a physical job, and did not come in for follow-up for 4.5 months despite noting discomfort with activities. Patient 5 was reassessed 9 months post-op, received additional treatment, and then felt 90% better. Either mean result showed an increase from previous studies. For example, one study involved 7 shoulder surgeons who reviewed 53 rotator cuff repairs. Of the 53 patients in a younger patient population, the demographics included 44 (83%) with double row repairs and 36 (68%) with medium tears. The mean post-operative SANE score was 80.8 at a mean follow-up of 35 months. See, Emery C. Lin et al., “Arthroscopic Primary Rotator Cuff Repairs in Patients Aged Younger Than 45 Years,” Arthroscopy, May 2013 Volume 29, Issue 5, Pages 811-817.

What is claimed is:

1. A method of attaching soft tissue to bone, comprising:
inserting a first anchor into bone, wherein the first anchor comprises three or more suture limbs extending therefrom that are coupled to the first anchor, wherein at least three of the three or more suture limbs coupled to the first anchor are fixed relative to the first anchor;
passing the at least three suture limbs through a portion of soft tissue, wherein at least one of the at least three suture limbs is passed through the portion of soft tissue at a first location that is different from a second location where one or more of the other suture limbs are passed through the portion of the soft tissue; and
securing the at least three suture limbs to one or more additional anchors that are positioned beyond an edge of the portion of soft tissue.

2. The method of claim 1, further comprising:
inserting a second anchor into the bone, wherein the second anchor comprises three or more suture limbs extending therefrom that are coupled to the second anchor, wherein at least three of the three of more suture limbs are fixed relative to the second anchor;
passing the at least three suture limbs through the portion of soft tissue, wherein at least one of the suture limbs is passed through the portion of soft tissue at a third location different from a fourth location where one or more of the other suture limbs are passed through the portion of the soft tissue; and
securing the at least three suture limbs coupled to the second anchor to the one or more additional anchors that are positioned beyond the edge of the portion of soft tissue.

3. The method of claim 1, wherein inserting the first anchor into bone includes inserting the first anchor in a footprint area under a soft tissue tear.

4. The method of claim 2, wherein inserting the first anchor into bone and inserting the second anchor into bone includes inserting the first anchor and second anchors in a footprint area under a soft tissue tear.

5. The method of claim 1, wherein passing the at least three suture limbs coupled to the first anchor through a portion of soft tissue includes passing the suture limbs through an underside of the portion of soft tissue.

6. The method of claim 2, wherein passing the at least three suture limbs coupled to the first anchor through a portion of soft tissue and passing the at least three suture limbs coupled to the second anchor through a portion of soft tissue includes passing the suture limbs through an underside of the portion of soft tissue.

7. The method of claim 1, wherein each of the at least three of the suture limbs coupled to the first anchor are passed through the portion of soft tissue at different locations.

8. The method of claim 2, wherein each of the at least three of the suture limbs coupled to the first anchor and each of the at least three of the suture limbs coupled to the second anchor are passed through the portion of soft tissue at different locations from each other.

9. The method of claim 1, wherein no knots are tied above the portion of soft tissue.

10. The method of claim 2, wherein no knots are tied above the portion of soft tissue.

11. The method of claim 1, wherein securing the at least three suture limbs coupled to the first anchor to the one or more additional anchors that are positioned beyond the edge of the portion of soft tissue includes securing the at least three suture limbs to the one or more additional anchors in a knot-less fashion.

12. The method of claim 2, wherein securing each of the suture limbs to one or more additional anchors that are positioned beyond the edge of the portion of soft tissue includes securing each of the suture limbs to the one or more additional anchors in a knot-less fashion.

13. A method of attaching soft tissue to bone, comprising:
inserting a first anchor into bone at a position that will be underneath at least a portion of soft tissue after completion of the method, the first anchor having three or more suture limbs extending therefrom, the portion of soft tissue having a top side and an underside;
fixing at least three lengths of suture relative to the first anchor after or while the anchor is inserted into the bone;
passing the at least three suture lengths up through the underside of the soft tissue portion, wherein each of the at least three suture lengths pass through different locations through the underside of the soft tissue portion; and
securing the at least three suture lengths to one or more additional anchors positioned beyond an edge of the soft tissue such that the at least three suture lengths contact the top side of the soft tissue portion and apply a downward force on the top side to secure the soft tissue portion in place.

14. The method of claim 13, fixing at least three lengths of suture relative to the first anchor after or while the anchor is inserted into the bone comprises tying a knot at a position that will be underneath at least a portion of soft tissue after completion of the method.

15. The method of claim 13, wherein no knots are tied above the top side of the portion of soft tissue.

16. The method of claim 13, wherein securing the at least three suture lengths to one or more additional anchors positioned beyond an edge of the soft tissue includes securing each of the suture limbs to the one or more additional anchors in a knot-less fashion.

17. A method of attaching soft tissue to bone, comprising:
inserting a first anchor into bone at a position that will be underneath at least a portion of soft tissue after completion of the method, the first anchor having three or more suture limbs extending therefrom, the portion of soft tissue having a top side and an underside;
fixing at least three lengths of suture relative to the first anchor after or while the anchor is inserted into the bone;
passing the at least three suture length up through the underside of the soft tissue portion, wherein each of the at least three suture lengths pass through different locations through the underside of the soft tissue portion;
securing the at least three suture lengths to a second anchor positioned beyond an edge of the soft tissue such that the at least three suture lengths contact the top side of the soft tissue portion and apply a downward force on the top side to secure the soft tissue portion in place;
inserting a third anchor into bone at a position that will be underneath at least a portion of soft tissue after completion of the method, the third anchor having three or more suture limbs extending therefrom;
fixing at least three lengths of suture relative to the third anchor after or while the third anchor is inserted into the bone;
passing the at least three suture length up through the underside of the soft tissue portion, wherein each of the at least three suture lengths pass through different locations through the underside of the soft tissue portion; and
securing the at least three suture lengths to a fourth anchor positioned beyond an edge of the soft tissue such that the at least three suture lengths contact the top side of the soft tissue portion and apply a downward force on the top side to secure the soft tissue portion in place.

18. The method of claim 17, fixing at least three lengths of suture relative to the first or second anchor comprises tying a knot at a position that will be underneath at least a portion of soft tissue after completion of the method.

19. The method of claim 17, wherein no knots are tied above the top side of the portion of soft tissue.

20. The method of claim 17, wherein securing the suture lengths to the second and fourth anchors includes securing each of the suture limbs to the second and fourth anchors in a knot-less fashion.
